(19) United States
(12) Patent Application Publication
(10) Pub. No.: US 2013/0103044 A1
(43) Pub. Date: Apr. 25, 2013

(54) Cervical Stretch Band

(71) Applicant: Sybil Brown, Indianapolis, IN (US)

(72) Inventor: Sybil Brown, Indianapolis, IN (US)

(21) Appl. No.: 13/590,454

(22) Filed: Aug. 21, 2012

Related U.S. Application Data
(60) Provisional application No. 61/550,031, filed on Oct. 21, 2011.

(51) Int. Cl. A61B 17/42

(52) U.S. Cl. 606/119

(57) ABSTRACT

A cervical stretch band device is disclosed that is designed for securing the neck of a woman's cervix tighter to prevent miscarriages and premature births. The cervical stretch band device comprises a band, a fastening component secured to a first end of the band, and at least one opening positioned on a second end of the band for receiving the fastening component. The first end and the second end of the band are secured together via the fastening component, wherein the fastening component is placed and locked within one of the openings. Once secured together, the band forms an opening to receive a neck of a cervix. If continuous pressure is placed on the band to expand it, the fastening component will be ejected from the opening, causing the band to detach and dislodge from the neck of the cervix, without causing any damage to the woman's cervix.

### CROSS-REFERENCE

This application claims priority from Provisional Patent Application Ser. No. 61/550,031 filed Oct. 21, 2011.

### BACKGROUND

Pregnant women, women with cervical incompetence, or women with a history of miscarriages, among others, may need to have the neck of their cervix held tightly together to help prevent miscarriages. If the cervix is left as is, the women may go into premature labor, need surgery to close the cervix, or their amniotic sac may become infected. However, some devices on the market for holding the cervix together may be dangerous for women, as they have to be surgically implanted and/or removed. Additionally, if the woman goes into labor and does not make it to a hospital in time, her cervix could rupture and/or become damaged. An effective solution is necessary.

There is a need for an improved cervical procedure for women that can hold the neck of a woman's cervix tighter to prevent miscarriages. The present invention comprises a one-piece, semi-stretchable band in which the ends can attach together. Further, if continuous pressure is placed on the band to expand it, the band can detach and dislodge itself, without causing any damage to the woman's cervix. Thus, by using this device, women can prevent miscarriages and premature births while lowering the risk of complications that occur with cervical suture surgery. Accordingly, women who are pregnant, women with cervical incompetence, or women who have a history of miscarriages can all benefit from the use of this device.

### SUMMARY

The following presents a simplified summary in order to provide a basic understanding of some aspects of the disclosed innovation. This summary is not an extensive overview, and it is not intended to identify key/critical elements or to delineate the scope thereof. Its sole purpose is to present some concepts in a simplified form as a prelude to the more detailed description that is presented later.

The subject matter disclosed and claimed herein, in one aspect thereof, comprises a cervical stretch band device that is designed for securing the neck of a woman's cervix tighter to prevent miscarriages and premature births. The cervical stretch band device comprises a band comprising a first end and a second end. A fastening component is secured to the first end of the band, and at least one opening is positioned on the second end of the band for receiving the fastening component. The first end and the second end of the band are secured together via the fastening component, wherein the fastening component is placed and locked within one of the openings. Once secured together, the band forms an opening to receive a neck of a cervix.

In a preferred embodiment, the band is a one-piece, semi-stretchable band that is generally flat. Furthermore, the band can comprise reinforced edges and/or reinforced ribbing to prevent breakage of the band and to provide for additional support. The band is tightened via the fastening component engaging another opening, to constrict the neck of the cervix tightly together. If continuous pressure is placed on the band to expand it, the fastening component will be ejected from the opening, causing the band to detach and dislodge from the neck of the cervix, without causing any damage to the woman's cervix.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

FIG. 1 illustrates a perspective view of a cervical stretch band device in accordance with the disclosed architecture.

FIG. 2 illustrates a perspective view of multiple cervical stretch band devices in various sizes in accordance with the disclosed architecture.

FIG. 3 illustrates a perspective view of the cervical stretch band device with the ends secured together in accordance with the disclosed architecture.

FIG. 4 illustrates a perspective view of the cervical stretch band device in use in accordance with the disclosed architecture.

FIG. 5 illustrates a perspective view of the cervical stretch band device in use, secured around a neck of a cervix in accordance with the disclosed architecture.

### DESCRIPTION OF PREFERRED EMBODIMENTS

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

The present invention discloses a cervical stretch band device that is designed for securing the neck of a woman's cervix tighter to prevent miscarriages and premature births. The cervical stretch band device comprises a band, a fastening component secured to a first end of the band, and at least one opening positioned on a second end of the band for receiving the fastening component. The first end and the second end of the band are secured together via the fastening component, wherein the fastening component is placed and locked within one of the openings. Once secured together, the band forms an opening to receive a neck of a cervix.

Furthermore, if continuous pressure is placed on the band to expand it, the fastening component will be ejected from the opening, causing the band to detach and dislodge from the neck of the cervix, without causing any damage to the woman's cervix. Thus, by using this device, women can prevent miscarriages and premature births while lowering the risk of complications that occur with cervical suture surgery. Accordingly, women who are pregnant, women with cervical incompetence, or women who have a history of miscarriages can all benefit from the use of this device.

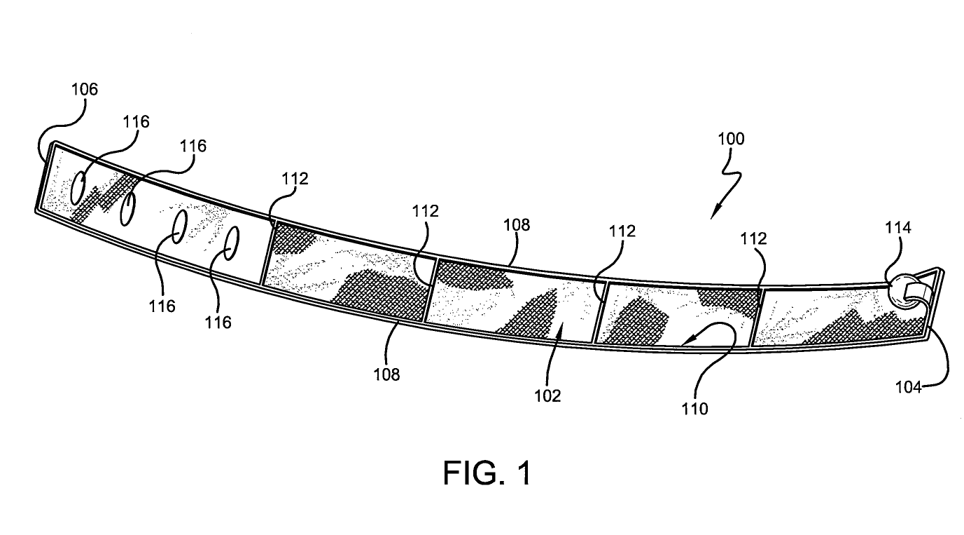

Referring initially to the drawings, FIG. 1 illustrates a cervical stretch band device 100 that is designed for securing the neck of a woman's cervix tighter to prevent miscarriages and premature births. The cervical stretch band device 100 comprises a band 102 comprising a first end 104 and a second end 106. Typically, the band 102 is rectangular in shape, however any other suitable shape can be used as is known in the art without affecting the overall concept of the invention.

The band 102 would generally be constructed of elastic, cotton, nylon, rubber, or a plastic, such as polyether ether ketone (PEEK), though any other suitable elastic material that is feasible for the inner body may be used to manufacture the band 102 as is known in the art without affecting the overall concept of the invention. The band 102 is approximately between 2 and 5 inches long as measured from the first end 104 to the second end 106, approximately between ¼^{th} and 1 inch wide as measured between sides 108, and approximately between 1/16^{th} and ¼^{th} of an inch thick.

Furthermore, the band 102 is a one-piece, semi-stretchable band that is generally flat. However, the band 102 can be comprised of multiple sections mated together instead of an integral unit, without affecting the overall concept of the invention. The band 102 can also be hypoallergenic, anti-bacterial, and/or moisture resistant depending on the wants and needs of a user. Additionally, the band 102 can comprise reinforced edges 110 and/or reinforced ribbing 112 to prevent breakage of the band 102 and to provide for additional support. Further, the bands 102 can be manufactured in various sizes and thicknesses depending on the wants and needs of a user.

Additionally, the band 102 comprises a latching or fastening component 114 secured to the first end 104 of the band 102. The latching or fastening component 114 is a latch hook, a ratcheting type clasp, a button, etc., or any other suitable fastening device as is known in the art. The fastening component 114 would generally be constructed of metal, such as titanium, cobalt chrome, or stainless steel, or a plastic, such as polyether ether ketone (PEEK), though any other suitable material that is feasible for the inner body may be used to manufacture the fastening component 114 as is known in the art without affecting the overall concept of the invention.

Furthermore, at least one opening 116 is positioned on the second end 106 of the band 102 for receiving the fastening component 114. Typically, there are two to four openings 116 for receiving the fastening component 114. The openings 116 comprise notches, slits, or other suitable openings as is known in the art. The first end 104 and the second end 106 of the band 102 are secured together via the fastening component 114, wherein the fastening component 114 is placed and locked within one of the openings 116. Once secured together, the band 102 forms an opening (not shown) to receive a neck of a cervix (not shown). The band 102 is then tightened via the fastening component 114 engaging another opening 116, to constrict the neck of the cervix tightly together. The band 102 acts to secure the neck of a woman's cervix tighter to prevent miscarriages or premature births. If continuous pressure is placed on the band 102 to expand it, the band 102 will detach and dislodge from the neck of the cervix, without causing any damage to the woman's cervix.

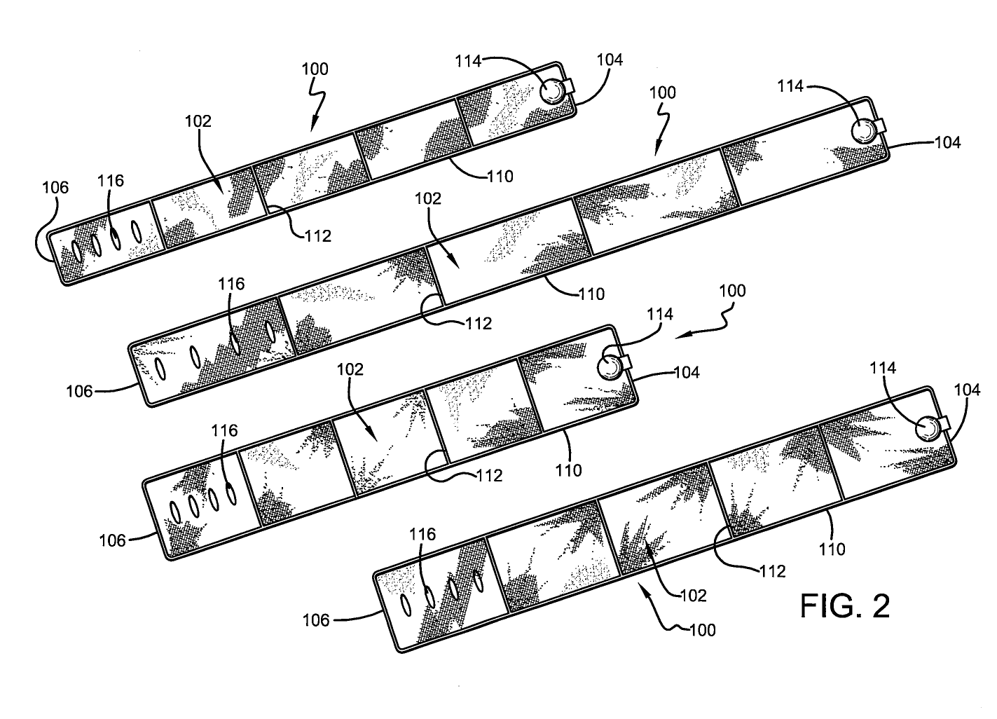

FIG. 2 illustrates multiple cervical stretch band devices 100 in various sizes. The cervical stretch band device 100 comprises a band 102 comprising a first end 104 and a second end 106. The first end 104 and the second end 106 of the band 102 are secured together via a fastening component 114. Once secured together, the band 102 forms an opening to receive a neck of a cervix.

Additionally, the band 102 is a one-piece, semi-stretchable band that is generally flat. However, the band 102 can be comprised of multiple sections mated together instead of an integral unit, without affecting the overall concept of the invention. Further, the surface of the band 102 can be treated, such that the band 102 is hypoallergenic and/or moisture resistant depending on the wants and needs of a user. Additionally, the band 102 may have antibacterial and/or antimicrobial properties inherent in the material or added as a coating. Furthermore, the band 102 can comprise reinforced edges 110 and/or reinforced ribbing 112 to prevent breakage of the band 102 and to provide for additional support. The reinforced edges 100 and/or the reinforced ribbing 112 may also act to retain the band 102 around the neck of the cervix and prevent slippage of the band 102 from the cervix. Additionally, the bands 102 can be manufactured in various sizes and thicknesses (as shown in FIG. 2) depending on the wants and needs of a user.

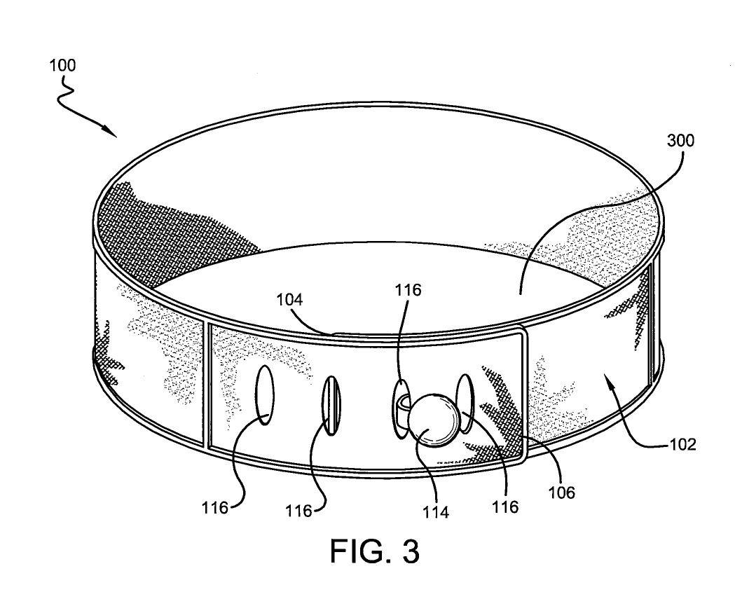

FIG. 3 illustrates the cervical stretch band device 100 with the ends 104 and 106 secured together via a fastening component 114. The band 102 comprises a fastening component 114 secured to the first end 104 of the band 102. The fastening component 114 is typically a latch hook (as shown in FIG. 3), but can also be a ratcheting type clasp, a button, etc., or any other suitable fastening device as is known in the art. The fastening component 114 would generally be constructed of metal, such as titanium, cobalt chrome, or stainless steel, or a plastic, such as polyether ether ketone (PEEK), though any other suitable material that is feasible for the inner body may be used to manufacture the fastening component 114 as is known in the art without affecting the overall concept of the invention.

Furthermore, at least one opening 116 is positioned on the second end 106 of the band 102 for receiving the fastening component 114. Typically, there are two to four openings 116 for receiving the fastening component 114. The openings 116 comprise notches, slits, or other suitable openings as is known in the art. The first end 104 and the second end 106 of the band 102 are secured together via the fastening component 114, wherein the fastening component 114 is placed and locked within one of the openings 116. Once secured together, the band 102 forms an opening 300 to receive a neck of a cervix (not shown). The band 102 is then tightened via the fastening component 114 engaging another opening 116, to constrict the neck of the cervix tightly together. Thus, the band 102 acts to secure the neck of a woman's cervix tighter to prevent miscarriages or premature births. However, if continuous pressure is placed on the band 102 to expand it, the fastening component 114 will be ejected from the opening 116, causing the band 102 to detach and dislodge from the neck of the cervix, without causing any damage to the woman's cervix. Furthermore, after a woman has safely reached her gestation period of 36-40 weeks, then the band 102 can be safely removed by a physician or other trained medical specialist. Typically, the cervical stretch band device 100 is secured to and/or removed from the cervix by a trained physician or other trained medical specialist via the use of forceps, clamps, or other medical instruments as is known in the art. Thus, the cervical stretch band device 100 can be easily secured around a neck of a woman's cervix and then removed, without causing damage to the cervix.

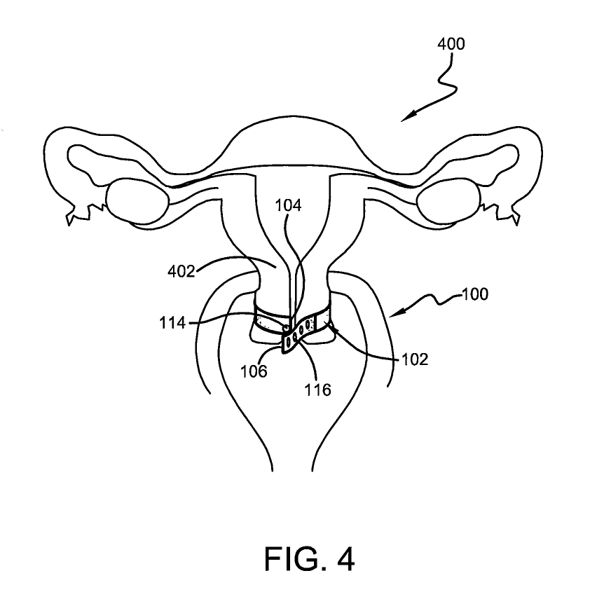

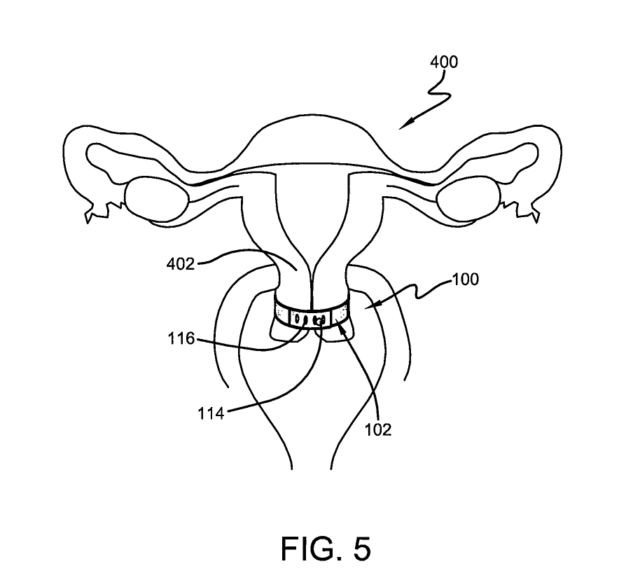

FIGS. 4-5 illustrate the cervical stretch band device 100 in use. In operation, a physician or other trained medical specialist would select a cervical stretch band device 100 in the desired length and thickness for the user. The physician would then insert the cervical stretch band device 100 into a user's cervix 400. Typically, the cervical stretch band device 100 is inserted into the cervix 400 via the use of forceps, clamps, or other medical instruments as is known in the art.

The physician would then secure the cervical stretch band device 100 together around the neck 402 of the cervix 400. Specifically, the first end 104 and the second end 106 of the band 102 are secured together via the fastening component 114, wherein the fastening component 114 is placed and locked within one of the openings 116. Once secured together, the band 102 can be tightened around the neck 402 of the cervix 400. The band 102 is tightened via the fastening component 114 engaging another opening 116, to constrict the neck 402 of the cervix 400 tightly together.

Once secured, the band 102 acts to secure the neck of a woman's cervix tighter to prevent miscarriages or premature births. However, if continuous pressure is placed on the band 102 to expand it, the fastening component 114 will be ejected from the opening 116, causing the band 102 to detach and dislodge from the neck 402 of the cervix 400, without causing any damage to the woman's cervix. Furthermore, after a woman has safely reached her gestation period of 36-40 weeks, then the band 102 is safely removed by a physician or other trained medical specialist. Typically, the cervical stretch band device 100 is removed from the cervix 400 via the use of forceps, clamps, or other medical instruments as is known in the art.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A cervical stretch band device comprising:
a band comprising a first end and a second end;
a fastening component secured to the first end of the band; and
at least one opening positioned on the second end of the band for receiving the fastening component; and
wherein the first end and the second end of the band are secured together via the fastening component to form an opening to receive a neck of a cervix.

2. The cervical stretch band device of claim 1, wherein the band is a one-piece, semi-stretchable band.

3. The cervical stretch band device of claim 2, wherein the band is flat.

4. The cervical stretch band device of claim 3, wherein the band is approximately between 2 and 5 inches in length.

5. The cervical stretch band device of claim 1, wherein the band is made of nylon, rubber, or plastic.

6. The cervical stretch band device of claim 1, wherein the band is hypoallergenic.

7. The cervical stretch band device of claim 1, wherein the band is anti-bacterial.

8. The cervical stretch band device of claim 1, wherein the band is moisture resistant.

9. The cervical stretch band device of claim 1, wherein the band further comprises reinforced edges.

10. The cervical stretch band device of claim 1, wherein the fastening component comprises a latch hook.

11. A cervical stretch band device comprising:
a band comprising a first end and a second end;
a latch hook secured to the first end of the band; and
at least one opening positioned on the second end of the band for receiving the latch hook; and
wherein the first end and the second end of the band are secured together via the latch hook to form an opening to receive a neck of a cervix.

12. The cervical stretch band device of claim 11, wherein the band is approximately between 2 and 5 inches in length.

13. The cervical stretch band device of claim 11, wherein the band is made of nylon, rubber, or plastic.

14. The cervical stretch band device of claim 11, wherein the band is hypoallergenic.

15. The cervical stretch band device of claim 11, wherein the band is anti-bacterial.

16. The cervical stretch band device of claim 11, wherein the band is moisture resistant.

17. A cervical stretch band device comprising:
a one-piece, semi-stretchable band comprising a first end and a second end;
a latch hook secured to the first end of the band; and
at least one opening positioned on the second end of the band for receiving the latch hook; and
wherein the first end and the second end of the band are secured together via the latch hook to form an opening to receive a neck of a cervix.

18. The cervical stretch band device of claim 17, wherein the band is made of nylon, rubber, or plastic.

19. The cervical stretch band device of claim 17, wherein the band is approximately between 2 and 5 inches in length.

20. The cervical stretch band device of claim 17, wherein the band is hypoallergenic.
